(19) United States
(12) Patent Application Publication
(10) Pub. No.: US 2009/0104689 A1
(43) Pub. Date: Apr. 23, 2009

(54) Microchip For Use In Cytometry, Velocimetry And Cell Sorting Using Polyelectrolytic Salt Bridges

(76) Inventors: Hee-Chan Kim, Seoul (KR); Taek Dong Chung, Seoul (KR); Hong Gu Chun, Seoul (KR)

Correspondence Address: HOFFMANN & BARON, LLP, 6900 JERICHO TURNPIKE, SYOSSET, NY, 11791, US

(21) Appl. No.: 11/791,648

(22) PCT Filed: Nov. 25, 2005
(86) PCT No.: PCT/KR05/03988

§ 371 Date: May 25, 2007

(30) Foreign Application Priority Data
Nov. 25, 2004 (KR) 10-2004-0097332

(51) Int. Cl. C12M 1/00; G01N 27/00

(52) U.S. Cl. 435/287.1; 422/82.01

(57) ABSTRACT

The present invention relates to a microchip using polyelectrolyte salt bridge for cytometry, velocimetry, and cell sorting. The microchip comprises; a) an inlet for solution to be analyzed, b) a microchannel which provides a moving passage for solution to be analyzed, c) at least one outlet for solution to be analyzed which has passed through the moving passage, d) at least one electrode system comprising a first and a second salt bridges connected to the microchannel (the two salt bridges face each other), and a first and a second reservoirs connected to said each salt bridge (the reservoir comprises electrode and standard electrolyte solution). The microchip detects analytes in the solution to be analyzed (for example, a cell) by detecting change of impedance. In detail, anion in the standard electrolyte solution, which is comprised in the first reservoir, moves from the first salt bridge to the second salt bridge across the microchannel. Impedance change occurs by interference of anion moving across the microchannel and the change can be detected by impedance analyzer connected to electrodes in the first and the second reservoirs.

### TECHNICAL FIELD

The present invention relates to a microchip. More particularly, the present invention relates to a microchip for use in cytometry, velocimetry and cell sorting, comprising polyelectrolytic salt bridges.

### BACKGROUND ART

The modern concept of micro total analysis systems (μTAS) is dated back to early 1990s when capillary electrophoresis (CE) was developed on a glass chip by Manz et al.¹ As previously well-documented, chemical and biological processes on microchip propose appreciable benefits that were unattainable with macro scale process configurations; tiny sample volume for analysis, low cost, easy automation, and high throughput by parallel processing². Taking these advantages, μTAS keeps expanding its applications to many different areas; clinical diagnostics^{3,4}, single chip Polymerase Chain Reaction (PCR)^{5,6}, DNA separation^{7,8}, DNA sequencing^{9,10}, biological and chemical analysis¹¹⁻¹³, cell analysis¹⁴⁻¹⁶ and flow cytometry¹⁶⁻²⁷.

Flow cytometry is a technology to measure some properties of cells as they move or flow in liquid suspension. The miniaturization of flow cytometry system for a point-of-care test (POCT) has great importance for not only cell biological research but also clinical uses, which include the stem cell separation from peripheral blood and control of the white blood cell (WBC) level for juvenile leukemia patients. In most flow cytometers, cells traveling in the interrogation region are detected by either optical or electrical method. Fluorescent activated cell sorting (FACS) adopts the former and Coulter counter is based on the latter²⁸. There have been many reports on microchip-based flow cytometers employing both detection methods. For instance, Wolff et al. presented a highly integrated chip for high-throughput FACS²¹. Ayllife et al. reported chip-based electrical analyzer operated by microchannel impedance spectroscopy¹⁷. In spite of the significant advances reported on FACS on microchip^{16,22-26} and electrical counter¹⁸⁻²⁰, flow cytometers on microchips are partially successful in practical applications yet.

Combining conventional FACS concept with microfluidic chip obviously aims miniaturization and simplification of the device doing similar jobs to those the currently commercialized large systems do. However, this idea has a few serious problems. Firstly, it requires cell modification by markers or antibodies, which may lead to alteration of the system under study. Secondly, optical parts can hardly be reduced to the size as small as the microchip itself. Even if fluidic parts become substantially small by introducing microfluidic technology, the whole system including other parts like the detection unit is still too large to be a practical device for POCT. Thirdly, the equipments for detection are rather expensive and complex to operate. Optical equipments are rarely as cheap as electronic devices and necessarily require fine alignment. Electrical detection method has been considered as an alternative to the optical technology in this regard. Electronic device is the most probable choice in terms of miniaturization, simplification and cost-effectiveness, assuming that it works as well as optical setup. But there are fundamental challenges for the electrical method to accomplish such good cytometric functions as the optical detection in FACS offers.

Theoretical calculation says that two electrodes should face to each other at opposite sides in the channel wall to obtain the best sensitivity and precision of impedance response¹⁸. There have been tried to overlay two glasses with planar metal band electrodes to be faced to each other²⁰ and to electroplate conducting metals on two planar electrodes, between which there is a microchannel¹⁷. Unfortunately, those trials made only limited success because of the following reasons. First of all, fabrication of such electrodes was tricky. Even if those are made somehow, it is hard to guarantee the reproducible geometry and characteristics as electrodes. Another problem is related to the electrode material and the frequency applied. Since the electrical property of a cell membrane is close to that of a capacitor, the impedance signal should be inversely proportional to the frequency applied. This tells that lower frequency generates larger change in impedance. The most desirable frequency is zero, namely DC signal. However, metal electrodes are not compatible to DC or low frequency of electric potential bias. Since electric double layer and/or faradaic reaction on the metal electrode surfaces intervenes the circuit, the impedance changes due to the cells become insignificant. As long as conventional metal electrodes are used, it must choose AC input at a high frequency, which makes cell detection less sensitive. In addition, the data for cell size reportedly correlate with the velocity. That is why calibration process with respect to velocity was suggested for the estimation of cell size^{18,22}.

In terms of cell sorting, there is one more challenge that should be addressed. In FACS, the moving cells are two dimensionally focused hydrodynamically so that the velocities are uniform within a limited error. That makes effective cell sorting possible in FACS. However one dimensional (horizontal) focusing on microfluidic chip cannot produce as good flow as generated in the conventional FACS system. Thus it is harder to obtain the uniform velocity of the cells on a microfluidic chip. The variation of velocity may generate pulses in wrong timing to push or pull the cell to be sorted. Considering the fact that the fast velocimetry of cells on microchips is one of the critical issues toward automatic cell-sorting on a microfluidic chip with high throughput, quick and accurate velocimetry of moving cells on the spot could help greatly in sorting cells in a flow cytometer.

A simple method previously reported for velocity measurement of flow cells is the video image velocimetry²⁹, where the velocity is estimated by observing the displacement of the cell within a known time interval. However, it has limitations in accuracy and cost because the video frame interval obviously regulates its resolution. Shah convolution Fourier transform is another method to extract velocity information on microfluidic chip³⁰. In this method, a mask with a periodic array of slits modulates the excitation beam in space and the cells underneath the mask undergo spatially modulated excitation. Fourier transforming the modulated fluorescent signals produces data containing velocity information. The mask with periodic slits can be replaced by a waveguide beam splitter for the purpose of integration on the microchip³¹. Another method for velocity measurement is to use a time interval of fluorescent peaks from two adjacent areas excited by acousto-optic modulator (AOM)²². Both Shah convolution Fourier transform and AOM methods increase the complexity of instrument and calculation, and have limitations in miniaturization because of the space for the optical system integrated.

### DISCLOSURE OF INVENTION

### Technical Problem

In order to solve the above-identified disadvantages, there is provided fabrication and performance of a flow cytometric or velocimetric chip using polyelectrolytic salt bridge-based electrode (PSBE). The concept of salt bridge was not so common in microfluidic chip research. Khandurina and co-workers applied porous silicate film as a salt bridge for electrophoresis³². Y. Takamura et al.³³ and A. Brask et al.³⁴ developed the low-voltage cascade electro-osmotic pump based on salt bridges. However, polymer-based salt bridge has not been used as an electrode for the detection of moving cells. The PSBE can be easily fabricated at the microchannel walls and make it possible to implement DC impedance analysis. Furthermore, two pairs of the PSBEs separated by a fixed length in a microchannel provide the data revealing the velocity information of cells on the same chip. It is believed that PSBE can offer a new opportunity to accomplish both the size-selective detection and simultaneous velocimetry of cells flowing along a microchannel without large or complex peripheral setup.

### Technical Solution

According to a preferred embodiment of the present invention, there is provided a microchip, comprising: a) an inlet through which a sample solution is introduced; b) a micro-channel along which the sample solution moves; c) at least one outlet which discharges the sample solution passed through the micro-channel; and d) at least one electrode system comprising i) a first and a second polyelectrolytic salt bridges, each of which is oppositely connected to the micro-channel and ii) a first and a second reservoirs, each of which is connected to the first and the second polyelectrolytic salt bridges and houses an electrode and a standard electrolyte solution. In the microchip, the electrodes housed in the first and the second reservoirs are electrically connected to an impedance analyzer. Anions contained in the standard electrolyte solution move across the micro-channel from the first polyelectrolytic salt bridge to the second polyelectrolytic salt bridge, and the movement of the anions is interfered by the sample that passes through the micro-channel. The interference causes an impedance change, and the impedance change is detected by the impedance analyzer. Such an impedance change is dependent upon the size of the sample. As the size of the moving sample increases, the peak of the impedance change increases. Further, The microchip may comprises two electrode systems separated by a fixed length in the micro-channel to measure the velocity of the sample by combination of the fixed length with a time difference at which the impedance change has occurred at each of the two electrode systems.

According to another preferred embodiment of the present invention, there is provided a microchip, comprising: a) an inlet through which a sample solution containing two or more cells is introduced; b) a micro-channel along which the sample solution moves; c) two outlets which discharge the sample solution passed through the micro-channel, one being located on an extended line from the micro-channel and the other at out of the extended line; d) a first electrode system comprising i) a first and a second polyelectrolytic salt bridges, each of which is oppositely connected to the micro-channel and ii) a first and a second reservoirs, each of which is connected to the first and the second polyelectrolytic salt bridges and houses an electrode and a standard electrolyte solution; e) a second electrode system comprising i) a third and a fourth polyelectrolytic salt bridges, each of which is oppositely connected to the micro-channel and ii) a third and a fourth reservoirs, each of which is connected to the third and the fourth polyelectrolytic salt bridges and houses an electrode and a standard electrolyte solution; and f) a means for converting a moving direction of the sample solution. In the microchip, the cell of no concern is collected into the outlet located on an extended line from the microchannel and the cell of concern is into the outlet located at out of the extended line by the working of the means for converting a moving direction of the sample solution.

### Advantageous Effects

The fabrication technology for PSBE in μTAS was developed and the performance as a flow cytometry glass microchip was also evaluated. It was demonstrated that the developed PSBE could successfully substitute the metal electrode for the impedance analysis in microfluidic glass chip. The PSBEs were embedded on cytometry and velocimetry microchips, which were evaluated using both fluorescent microbeads and human blood cells. Test results show that screening rate over 3,000 samples s⁻¹, measurement of cell velocity up to 100 mm s⁻¹, and velocity-free classification by particle size are possible.

The PSBE of the present invention suggests many useful applications. For instance, electrochemical cells that need to be integrated on microchips or decoupler in chip-based electrophoresis for electrochemical detection will possibly enjoy this unit device. The PSBEs chip for both cell counting and velocimetry are practically suitable for miniaturization so as to be applicable to small-sized point of care testing (POCT) devices. The present technology will offer a chance toward future Microsystems for clinical uses, for example, a miniaturized stem cell collector.

### BRIEF DESCRIPTION OF THE DRAWINGS

FIG. 1 is a resolved perspective view showing a preferred embodiment of the microchip, in accordance with the present invention.

FIG. 2 is a combined perspective view of the microchip shown in FIG. 1.

FIG. 3 is a drawing showing a working principle of the microchip, in accordance with the present invention.

FIG. 4 is a resolved perspective view showing another preferred embodiment of the microchip, in accordance with the present invention.

FIG. 5 is a resolved perspective view showing further another preferred embodiment of the microchip, in accordance with the present invention.

FIG. 6 is a graph showing an impedance variation due to fluorescent microbeads moving through the microchannel.

FIG. 7 is a histogram of the peak amplitude of impedance change obtained using two kinds of fluorescent microbeads with 9.95□ and 5.70□ in diameter. The two groups are clearly separated. The peak amplitude alone clearly tells one group of microbeads from the other.

FIG. 8 is a scatter plot of the velocity and the peak amplitude obtained for two kinds of fluorescent microbeads, 9.95□ and 5.70□ in diameter. Two groups are clearly separated. The amplitude of impedance peak for each group is independent of flow velocity, so two different sized microbeads can be classified solely by the peak amplitude.

FIG. 9 is a scatter plot of the velocity and the peak amplitude for red blood cell and white blood cell moving along the microchannel on the velocimetry microchip.

### MODE FOR THE INVENTION

In the following, the present invention will be more fully illustrated referring accompanied drawings.

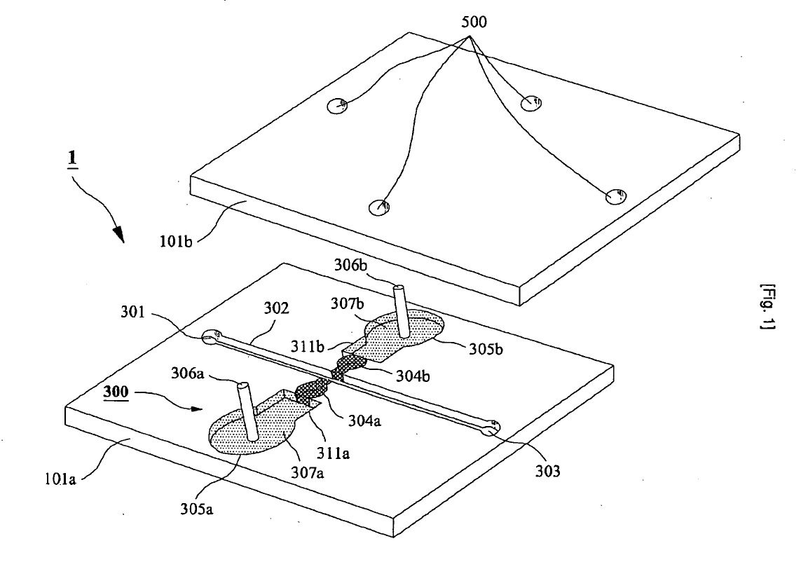

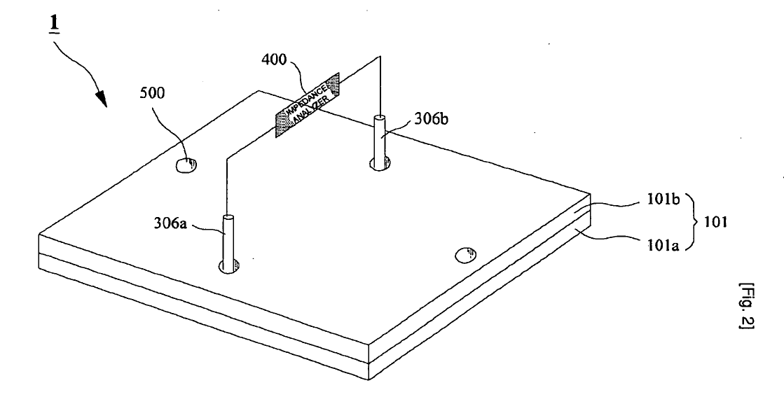

FIG. 1 is a resolved perspective view showing a preferred embodiment of the microchip, in accordance with the present invention and FIG. 2 is a combined perspective view of the microchip shown in FIG. 1.

As shown in FIG. 1, the microchip (1) of the present invention comprises a first and a second substrates (101a, 101b, totally “101”). On the first substrate (101a), an inlet (301) through which a sample solution is introduced, a micro-channel (302) along which the sample solution moves, an outlet (303) which discharges the sample solution passed through the micro-channel (302), and an electrode system (300) comprising a first and a second polyelectrolytic salt bridges (304a, 304b), each of which is oppositely connected to the micro-channel (302) and a first and a second reservoirs (305a 305b), each of which is connected to the first and the second polyelectrolytic salt bridges (304a, 304b) into which electrodes (306a, 306b) and standard electrolyte solutions (307a, 307b) are housed at each of the reservoirs (305a, 305b). In the microchip (1), the electrodes (306a, 306b) housed in the first and the second reservoirs (305a, 305b) are electrically connected to an impedance analyzer (400). By combining the first substrate (101a) with the second substrate (101b) into which holes (500) are formed in predetermined positions, the microchip (1) is fabricated.

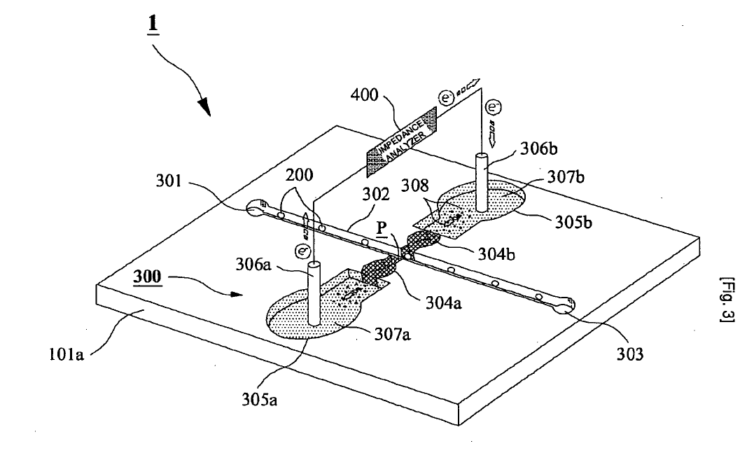

FIG. 3 is a drawing showing a working principle of the microchip, in accordance with the present invention. When a bias voltage is applied by the impedance analyzer (400) to the two electrodes (306a, 306b), anions (308) contained in the standard electrolyte solution (307b) move across the micro-channel (302) from the second polyelectrolytic salt bridge (304b) to the first polyelectrolytic salt bridge (304a). Thereafter, the anions undergo an oxidation reaction in contacts with the electrode (306a) of the reservoir (305a). In the reservoir (305b), cations (not shown) present in the standard electrolyte solution (307b) is reduced with aid of electrons (e⁻) delivered through the electrode (306b). By applying a constant bias voltage, the anions (308) may move in a constant rate across the micro-channel (302) from the second polyelectrolytic salt bridge (304b) to the first polyelectrolytic salt bridge (304a). In a meanwhile, samples (200) (for example, cells) contained in the sample solution introduced through the inlet (301) moves along the micro-channel (302). At an intersect point (P) between the polyelectrolytic salt bridges (304a, 304b) and the micro-channel (302), the samples (200) interferes with the movement of the anions (308). In order words, the movement of the anions (308) is interfered by the samples (200), which causes an impedance change. The impedance analyzer (400) located between the electrodes (306a, 306b) detects the impedance change. According to one specific embodiment of the present invention, the impedance change was proven to be dependent upon the size of a cell, irregardless of the velocity of a cell. In other words, the impedance change was found to selectively respond to the size of the cell. Further, as the size of the cell increases, the magnitude of the impedance change also increases. It is believed that such a change is resulted from the increased interference caused by a larger sized cell. As an impedance analyzer (400), a direct-current (DC) impedance analyzer or low frequency wave impedance analyzer may be used.

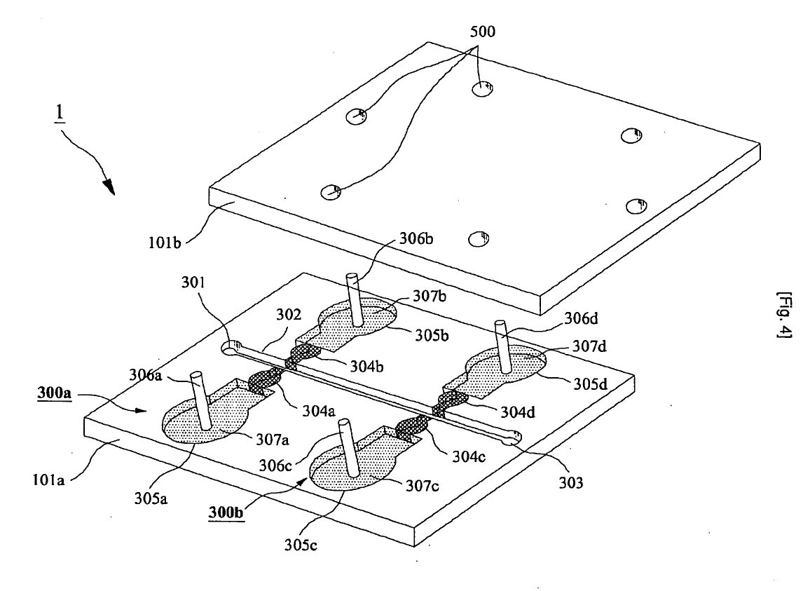

FIG. 4 is a resolved perspective view showing another preferred embodiment of the microchip, in accordance with the present invention. As shown in FIG. 4, the microchip (1) comprises two electrode systems (300a, 300b) separated by a fixed length. Specifically, the microchip (1) comprises, inside the substrates (101), an inlet (301) through which a sample solution is introduced, a micro-channel (302) along which the sample solution moves, an outlet (303) which discharges the sample solution passed through the micro-channel (302), a first electrode system (300a) comprising a first and a second polyelectrolytic salt bridges (304a, 304b), each of which is oppositely connected to the micro-channel 302 and a first and a second reservoirs (305a, 305b), each of which is connected to the first and the second polyelectrolytic salt bridges (304a, 304b) into which electrodes (306a, 306b) and standard electrolyte solutions (307a, 307b) are housed at each of the reservoirs (305a, 305b), and a second electrode system (300b) comprising a third and a fourth polyelectrolytic salt bridges (304c, 304d), each of which is oppositely connected to the micro-channel (302) and a third and a fourth reservoirs (305c, 305d), each of which is connected to the third and the fourth polyelectrolytic salt bridges (304c, 304d) into which electrodes (306c, 306d) and standard electrolyte solutions (307c, 307d) are housed at each of the reservoirs (305c, 305d).

The impedance changes are occurred at both of the first electrode system (300a) and the second electrode system (300b), as illustrated in FIG. 3. Herein, the first electrode system (300a) and the second electrode system (300b) are preferably connected each independently to two isolated impedance analyzers (400a, 400b), in order to suppress cross talk which may be caused from leakage current. The microchip shown in FIG. 4 detects the velocity of the moving sample. Specifically, by detecting a time difference between the impedance changes caused by interactions of the samples (200) with the first and the second electrode systems (300a, 300b) separated by a fixed length, the velocity of the moving sample can be revealed. The velocity of the moving sample is calculated referring the following formula:

v=d/ΔT=d/(T₂−T₁)

wherein, v represents the velocity of the moving sample, d represent the separated length between the first and the second electrode systems, and ΔT represents a time difference between a detection time (T₂) at which the impedance change is detected by the second impedance analyzer and a detection time (T₁) at which the impedance change is detected by the first impedance analyzer.

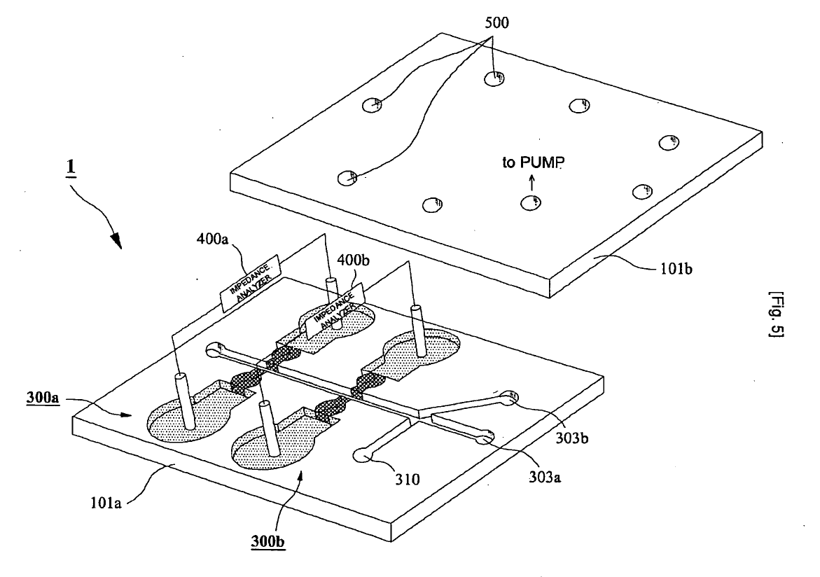

FIG. 5 is a resolved perspective view showing further another preferred embodiment of the microchip, in accordance with the present invention. As shown in FIG. 5, the microchip (1) of the present invention comprises an inlet (301) through which a sample solution is introduced, a micro-channel (302) along which the sample solution moves, two outlets (303a, 303b) which discharge the sample solution passed through the micro-channel (302), a first electrode system (300a) comprising a first and a second polyelectrolytic salt bridges (304a, 304b), each of which is oppositely connected to the micro-channel 302 and a first and a second reservoirs (305a, 305b), each of which is connected to the first and the second polyelectrolytic salt bridges (304a, 304b) into which electrodes (306a, 306b) and standard electrolyte solutions (307a, 307b) are housed at each of the reservoirs (305a, 305b), a second electrode system (300b) comprising a third and a fourth polyelectrolytic salt bridges (304c, 304d), each of which is oppositely connected to the micro-channel (302) and a third and a fourth reservoirs (305c, 305d), each of which is connected to the third and the fourth polyelectrolytic salt bridges (304c, 304d) into which electrodes (306c, 306d) and standard electrolyte solutions (307c, 307d) are housed at each of the reservoirs (305c, 305d), and a connection port (310) to which a pump (not shown) for converting a moving direction of the sample solution is connected. To the first electrode system (300a) and the second electrode system (300b), two isolated impedance analyzers (400a, 400b) are preferably connected each independently. The microchip system shown in FIG. 5 may be used in cell sorting. Detailed explanation is as follows. With aid of the peak amplitude of the impedance change detected by the first impedance analyzer (400a) or the second impedance analyzer (400b), the kind of a cell is identified. Suppose that two different cells having different sizes are contained in the sample solution. In this case, the peak amplitude of the first cell is distinguished from that of the second cell. Therefore, a time difference between a detection time (T₂) at which the impedance change is detected by the second impedance analyzer (400b) and a detection time (T₁) at which the impedance change can be measured with aid of the first impedance analyzer (400a). Based on the cell identified by the peak amplitude, in combination with the fixed length and a time elasped, the velocity of the moving cell can be revealed. Based on the velocity thus calculated, an estimated time for the sample to reach the position to which the pump is connected can be also measured. These data makes it possible to separate two different cells contained in the sample solution, with aid of the working of the pump. Specifically, based on the determined kind of the cell by the amplitude of the impedance peak and the velocity, it can be measured when the first cell reaches the position to which the pump connected. A control system (not shown) operates the pump (not shown) at that time, and by the working of the pump, the moving direction of the first cell is converted to the second outlet (303b). As thus, the first cell is collected into the second outlet (303b). In a similar manner, when the second cell is found to reach the position to which the pump connected, the pump does not work. The second cell that does not undergo change of the moving direction is collected into the first outlet (303a). As a result, the first cell and the second cell are selectively sorted.

Preferably, the first outlet (303a) is located on an extended line from the micro-channel (302), and the second outlet (303b) is located at out of the extended line from the micro-channel (302). Among the cells contained in the sample solution, the cell of concern undergoes change of the moving direction, by the working of the pump connected to the connection port (310), and is collected into the second outlet (303b). The pump is not operated to the cell of no concern, and thus, the cell of no concern is collected into the first outlet (303a). As a result, the cell of concern is selectively sorted from the sample solution containing two or more cells. If necessary, the number of the outlets (303) can be adjusted to that of the cells. Further, various means for converting the moving direction of the sample solution (for example, a valve) can be adopted as an alternative to the pump.

According to the preferred embodiment of the present invention, a metal/metal salt electrode is preferable as an electrode. As used herein, “metal/metal salt electrode” means an electrode comprising a metal core onto which a metal salt is coated onto the surface thereof. In addition, as an anion, a chloride ion (CF) gives satisfactory result. The polyelectrolytic salt bridge can be made of various polyelectrolytics that do not interfere with the movement of anions, independently of pH. Preferably, the polyelectrolytic salt bridges have an increased capacity to hold the anions. According to the specific example of the present invention, poly-diallyldimethylammonium chloride was used as a material for the polyelectrolytic salt bridge. According to the preferred specific embodiment of the present invention, the polyelectrolytic salt bridge has a calabash shape and the narrow side of the calabash is designed to contact the microchannel. This is to increase the sensitivity of the impedance change. The contact area can be suitably chosen regarding cell size, the anions to be used, the amplitude of the impedance peak, and so on. Further, between the polyelectrolytic salt bridge and the reservoir, a buffering region (denoted as 311a, 311b of FIG. 1) can be installed in order to prevent the polyelectrolytic salt bridge formed by photo-polymerization from invasion of the reservoir. Further, in the above, the microchip is formed by combination of the two substrates, the first substrate (101a) and the second substrate (101b). This is just an exemplary one. The microchannel network can be formed inside one substrate or, combination of three or more substrates. In addition, the substrate may be made from glass or polymer substrate, and tubes can be installed on the inlet and/or outlet in order to facilitate introduction and discharge of the sample solution.

### EXAMPLES

Microchip Fabrication

Corning 2947 precleaned slide glasses (75 mm by 25 mm, 1 mm thick) were used as substrates. A slide glass was cleaned in piranha solution (H₂SO₄:H₂O₂=3:1) for 1 h before washing the slide glass with deionized (DI) water (NANOpure Diamond, Barnstead, USA) and cleaned with acetone (CMOS grade, J. T. Baker, USA), methanol (CMOS grade, J. T. Baker, USA) and DI water twice sequentially. The cleaned slide glass was dehydrated on a 150° C. hot plate for 10 min and cooled down to room temperature. In order to modify the surface of the glass substrate, hydrophobic hexa methyl disilazane (HMDS) (Clariant, Switzerland) was spin-coated (Won corporation, Korea) at 4,000 rpm for 30 s on the slide glass, on which spin-coating of the photo resist (PR) of AZ5214-E (Clariant) was followed at 4,000 rpm for 30 s. After soft baking of photo-resist (PR) on a hot plate at 100° C. for 60 s, the slide glass was cooled down to room temperature and aligned under a pattern mask. Exposing to UV light (365 nm) with intensity of 16 mW cm⁻² for 6.5 s (MDE-4000, Midas, Korea) was followed by developing the PR with AZ300MIF (Clariant) for 45 s. And then the slide glass was washed with DI water and the PR was hard-baked on a hot plate at 105° C. for 15 min. HMDS and PR layers, which were spin-coated and baked in the same way as described above, protected the other side of the slide glass from the etching solution. The slide glass was etched with 6:1 buffered oxide etch solution (J. T. Baker) for 40 min at 25° C. The washing processes consist of a few successive steps; rinsing with DI water and acetone, sonicating in acetone for 5 min by ultrasonic cleaner (3510E-DTH, Bransonic, USA), soaking in methanol and DI water. Another flat slide glass that is to cover the etched glass was drilled at the positions for reservoirs with a diamond drill with 2 mm in diameter at 18,000 rpm. And then the flat side slide glass was cleaned in piranha solution for 1 h. The pairs of etched and flat slide glasses were permanently attached by thermal bonding. When two slide glasses contact each other for bonding, DI water filled between the slides keeps away from air bubbles. The glasses were heated up to 600° C. in a furnace (CRF-M15, CEBER, Korea) and temperature was maintained at 600° C. for 6 h, which was followed by slowly cooling down the furnace to room temperature for 10 h.

Polyelectrolytic Salt Bridge (PSBE) Fabrication.

Diallyldimethylammonium chloride (DADMAC) was selected for the material of salt bridge. 65% DADMAC aqueous solution was polymerized to yield poly-DADMAC (PDADMAC) by shedding UV light in the presence of 2 wt % photoinitiator and 2 wt % cross linker. The high charge density makes PDADMAC hold many anions inside the polymer structure so that the transport of the mobile anions is facile and the apparent resistance of the polymer plug decreases. Moreover the stationary charge of PDADMAC is independent of pH in the medium. Thus PDADMAC possesses good properties for a salt bridge. DADMAC, photo-initiator (2-hydroxy-4-(2-hydroxyethoxy)-2-methylpropiophenone), and cross-linker (N,N-Methylene-bisacrylamide) were purchased from Sigma-Aldrich (St. Louis, Mo., USA).

The salt bridge fabrication process using photopolymerization technique is as follows. The microchannel network of a microfluidic glass chip was filled with the DADMAC solution with the composition described above. The mask on the chip was aligned and subsequently exposed to UV light (365 nm) with intensity of 16 mW cm⁻² for 5.0 s. DADMAC monomers were polymerized to form three-dimensional PDADMAC, filling the calabash-shaped region shown in FIG. 1. After the photopolymerization, the microchannel was cleaned with 1 M KCl solution to remove the DADMAC monomers that remain. The dimension of the microchannel was 50□ wide and 22□ deep.

The Electrical Properties of the PSBEs.

The microchannel network of a microfluidic glass chip was filled with isotonic 0.92% NaCl solution. The two Ag/AgCl wires in corresponding reservoirs as shown in FIG. 1 were connected to an LCR meter (Precision Component Analyzer 6440A, Wayne Kerr, USA). The impedance between the two reservoirs of salt bridge was recorded as frequency continuously changes from DC to 3.0 MHz. Test results showed a flat impedance property of 30 kΩ throughout the whole frequency range.

Sample Preparation

The performance of the cytometry microchip with PSBEs was evaluated by the DC impedance analysis with fluorescent microbeads of 9.95□ (P(S/V-COOH), (480, 520), Bangs Laboratory, USA) and 5.70□ (P(S/5.5% DiVinylBenzene/5% MAA), (480, 520), Bangs Laboratory) in diameter. Fluorescent microbeads were diluted in isotonic NaCl solution to 0.025 wt % and 0.005 wt % for 9.95□ and 5.70□, respectively.

Red blood cells (RBC) and white blood cells (WBC) were sampled from a normal person and separated by centrifuge. RBC and WBC were diluted in RPMI 1640 medium (1×, Jeil biotechservices, Korea) to 0.0025 cells pL⁻¹ before being used in the experiment.

Signal Detection and Data Acquisition

Diluted fluorescent microbead solution was injected into the microchannel using a syringe pump (KDS100, KD Scientific, USA). FIG. 3 shows the configuration of the microfluidic glass chip on which DC impedance analysis was implemented. PSBEs were connected to an external DC impedance analyzer through isotonic NaCl solution and Ag/AgCl electrodes. 0.4 V DC bias generated about 13□ of DC current, which constantly maintained at least for 1 h without reversing the polarity. Experiments could be extended to longer than 1 h, if necessary, by just switching the bias polarity. The impedance varying signals that were generated when cells or microbeads went through the microchannel between PSBEs were amplified with a total gain of 2,000.

For the velocity estimation of moving cells, two pairs of the PSBEs were fabricated with the separation of 1 mm, which is shown in FIG. 4. Two isolated power supplies served the impedance analyzing circuits for the respective pair of PSBEs in order to suppress cross-talk between two pairs of the PSBEs. Two signal outputs from two impedance analyzing circuits are digitized with Lab-PC-1200 (National Instruments, Austin, Tex., USA) at sampling frequency of 30 kHz.

### RESULTS AND DISCUSSION

Detection of Moving Microbeads

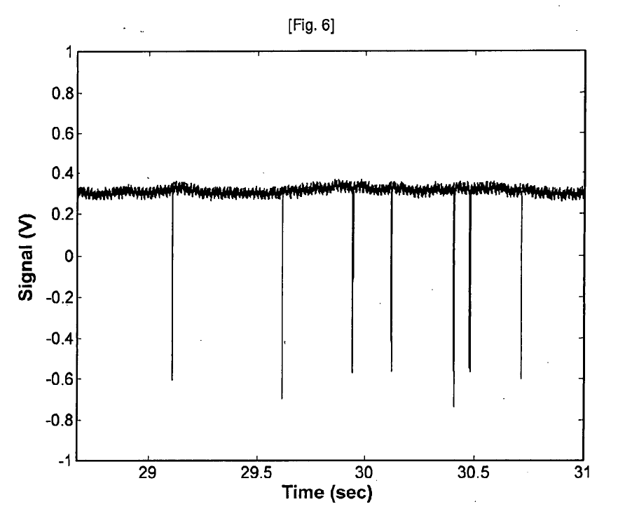

FIG. 6 shows an amplified signal of the impedance between the two PSBEs responding to 9.95□ fluorescent microbeads randomly passing along the microchannel. Each downward peak corresponds to a single microbead. The screening rate of the developed cytometry microchip can be estimated from the width of the peak signal for a cell passed through the detection volume between the PSBEs. The half-power widths of the signals revealed that the maximum screening rate is higher than 3,000 cells per second. The fast screening rate was partly attributed to the quick response of DC impedance analysis, which was possible only with the PSBEs. Parallel processing on a single microfluidic chip is expected to successfully raise the screening rate.

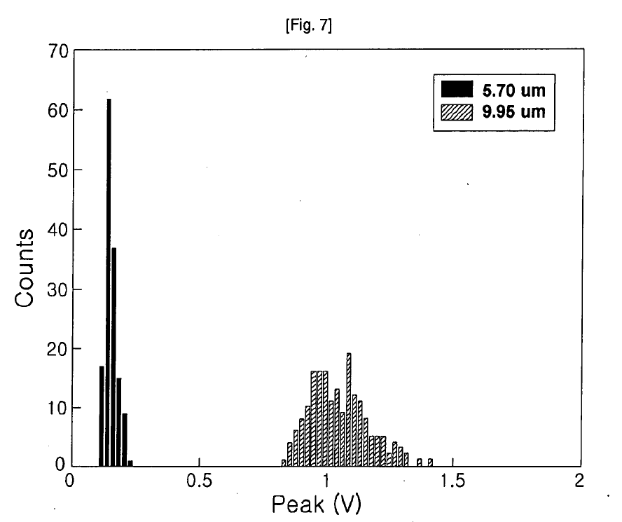

FIG. 7 shows the histogram of the peak amplitude for two kinds of fluorescent microbeads with different diameters. Correlation between peak height and the impedance was investigated using two kinds of fluorescent microbeads, 9.95□ and 5.70□. Test results showed that the distribution of peak amplitude was (m, σ²)=(1.0612, 0.1236²) for 9.95□ and (m, σ²)=(0.1635, 0.0263²) for 5.70□. A high frequency AC impedance analysis between co-planar metal electrodes generates the signals, the amplitudes of which are highly dependent on the altitude of the cell in the microchannel even when the flow in the microchannel network is hydrodynamically focused. However, DC impedance analysis using PSBEs gives the impedance signals that are not affected by the position of cells in interrogation region. As a result, the dependence of impedance signals on relative position from the electrodes can be markedly reduced so that problems stemming from the hydrodynamic focusing on microfluidic chip become less critical.

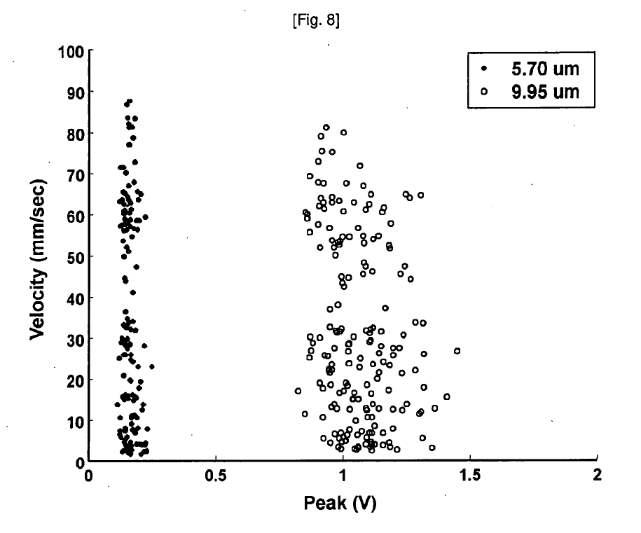

Two adjacent pair of the salt bridge electrodes pair illustrated in FIG. 4 will provide impedance peak signals with a certain time interval when a single cell successively passes through them. Velocity of a moving cell is calculated by dividing the fixed distance between two pairs of the salt bridge electrodes with the time elapsed. One pair of the salt bridge electrodes was away from the other by the distance of 1 mm, and the results were summarized in FIG. 8. As shown in FIG. 8, the peak amplitudes obtained for two kinds of fluorescent microbead, 9.95□ and 5.70□ in diameter were clearly separated. Further, the correlation between peak amplitude and flow velocity was fairly low. The Pearsons correlation value for each size was −0.244 and −0.207 for 9.95□ and 5.70□, respectively.

Counting Cells in Human Blood

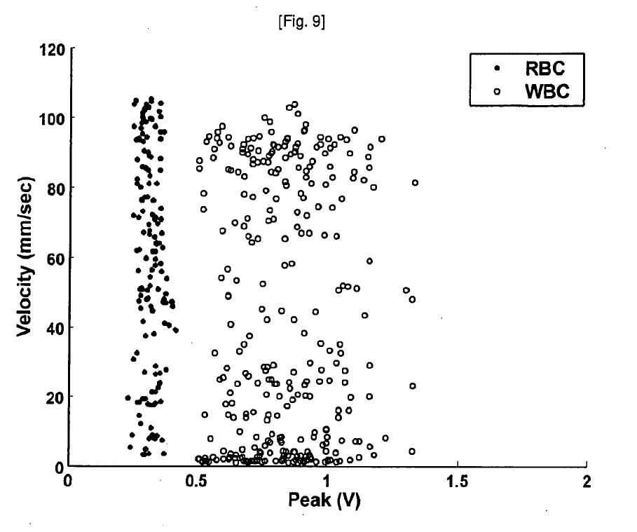

The performance of the developed cytometry microchip was evaluated with RBC and WBC from human blood sample. The size of the blood cell is distributed between 6-9□ and 12-18□ for RBC and WBC, respectively. According to the results from the experiments with microbeads, RBC and WBC should be able to be classified by the difference between their sizes. FIG. 9 displays a scatter plot of the velocity and the peak amplitude obtained from human blood cells. Test result showed that peak amplitude distribution is (m, σ²)=(0.3135, 0.03832) and (m, σ²)=(0.8319, 0.17922) for RBC and WBC, respectively. It is clear that the peak amplitude rarely correlate with the velocity in the range of 1 mm s⁻¹ to 100 mm s⁻¹. Thus it can be stated that the reliable classification of RBC and WBC is possible with the peak amplitude only.

The results from the experiments with human blood cells show that complete blood cell counting (CBC) with a hand-held device is possible on the spot. This means that the developed cytometry microchip is applicable to POCT type cell counter for many clinical applications including the WBC level control for juvenile leukemia patients.

### REFERENCES

(1) Manz, A.; Graber, N.; Widmer, H. M. Sensors and Actuators B-Chemical 1990, 1, 244.
(2) Erickson, D.; Li, D. Analytica Chimica Acta 2004, 507, 11-26.
(3) Tian, H. J.; Jaquins-Gerstl, A.; Munro, N.; Trucco, M.; Brody, L. C.; Landers, J. P. Genomics 2000, 63, 25-34.
(4) Pasas, S. A.; Lacher, N. A.; Davies, M. I.; Lunte, S. M. Electrophoresis 2002, 23, 759-766.
(5) Waters, L. C.; Jacobson, S. C.; Kroutchinina, N.; Khandurina, J.; Foote, R. S.; Ramsey, J. M. Analytical Chemistry 1998, 70, 5172-5176.
(6) Woolley, A. T.; Hadley, D.; Landre, P.; deMello, A. J.; Mathies, R. A.; Northrup, M. A. Analytical Chemistry 1996, 68, 4081-4086.
(7) Shi, Y. N.; Simpson, P. C.; Scherer, J. R.; Wexler, D.; Skibola, C.; Smith, M. T.; Mathies, R. A. Analytical Chemistry 1999, 71, 5354-5361.
(8) Ueda, M.; Kiba, Y.; Abe, H.; Arai, A.; Nakanishi, H.; Baba, Y. Electrophoresis 2000, 21, 176-180.
(9) Liu, S. R.; Shi, Y. N.; Ja, W. W.; Mathies, R. A. Analytical Chemistry 1999, 71, 566-573.
(10) Paegel, B. M.; Emmich, C. A.; Weyemayer, G. J.; Scherer, J. R.; Mathies, R. A., USA 2002; 574-579.
(11) Beebe, D. J.; Mensing, G. A.; Walker, G. M. Annual Review of Biomedical Engineering 2002, 4, 261-286.
(12) Jakeway, S. C.; de Mello, A. J.; Russell, E. L. Fresenius Journal of Analytical Chemistry 2000, 366, 525-539.
(13) Chovan, T.; Guttman, A. Trends in Biotechnology 2002, 20, 116-122.
(14) Stuart, J. N. Sweedler, J. V. Analytical and Bioanalytical Chemistry 2003, 375, 28-29.
(15) Lin, Y. C.; Jen, C. M.; Huang, M. Y.; Wu, C. Y.; Lin, X. Z. Sensors and Actuators B-Chemical 2001, 79, 137-143.
(16) Fu, A. Y.; Spence, C.; Scherer, A.; Arnold, F. H.; Quake, S. R. Nature Biotechnology 1999, 17, 1109-1111.
(17) Ayliffe, H. E.; Frazier, A. B.; Rabbitt, R. D. Journal of Microelectromechanical Systems 1999, 8, 50-57.
(18) Gawad, S.; Schild, L.; Renaud, P. Lab on a Chip 2001, 1, 76-82.
(19) Larsen, U. D.; Blankenstein, G., Chicago, Ill., Jun. 16-19, 1997; IEEE; 1319-1322.
(20) Gawad, S.; Batard, P.; Seger, U.; Metz, S.; Renaud, P. Micro total analysis systems 2002, 649-651.
(21) Wolff, A.; Perch-Nielsen, I. R.; Larsen, U. D.; Friis, P.; Goranovic, G.; Poulsen, C. R.; Kutter, J. P.; Telleman, P. Lab on a Chip 2003, 3, 22-27.
(22) Eyal, S.; Quake, S. R. Electrophoresis 2002, 23, 2653-2657.
(23) Sobek, D.; Young, A. M.; Gray, M. L.; Senturia, S. D., New York 1993; 219-224.
(24) Schrum, D. P.; Culbertson, C. T.; Jacobson, S. C.; Ramsey, J. M. Analytical Chemistry 1999, 71, 4173-4177.
(25) McClain, M. A.; Culbertson, C. T.; Jacobson, S. C.; Ramsey, J. M. Analytical Chemistry 2001, 73, 5334-5338.
(26) Kruger, J.; Singh, K.; O'Neill, A.; Jackson, C.; Morrison, A.; O'Brien, P. Journal of Micromechanics and Microengineering 2002, 12, 486-494.
(27) Altendorf, E.; Zebert, D.; Holl, M.; Vannelli, A.; Wu, C. C.; Schulte, T., Dordrecht, The Netherlands 1998; Kluwer Academic Publishers; 73-76.
(28) Durack, G.; Robinson, J. P. Emerging tools for single-cell analysis, 2000.
(29) Barker, S. L. R.; Ross, D.; Tarlov, M. J.; Gaitan, M.; Locascio, L. E. Analytical Chemistry 2000, 72, 5925-5929.
(30) Crabtree, H. J.; Kopp, M. U.; Manz, A. Analytical Chemistry 1999, 71, 2130-2138.
(31) Mogensen, K. B.; Kwok, Y. C.; Eijkel, J. C. T.; Petersen, N. J.; Manz, A.; Kutter, J. P. Analytical Chemistry 2003, 75, 4931-4936.
(32) Khandurina, J.; Jacobson, S. C.; Waters, L. C.; Foote, R. S.; Ramsey, J. M. Analytical Chemistry 1999, 71, 1815-1819.
(33) Takamura, Y.; Onoda, H.; Inokuchi, H.; Adachi, S.; Oki, A.; Horiike, Y. Kluwer Academic Publishers 2001, 230.
(34) Brask, A.; Goranovic, G.; Bruus, H. Sensors and Actuators B-Chemical 2003, 92, 127-132.

1. A microchip, comprising
a) an inlet through which a sample solution is introduced;
b) a micro-channel along which the sample solution moves;
c) at least one outlet which discharges the sample solution passed through the microchannel; and
d) at least one electrode system comprising
i) a first and a second polyelectrolytic salt bridges, each of which is oppositely connected to the micro-channel and
ii) a first and a second reservoirs, each of which is connected to the first and the second polyelectrolytic salt bridges and houses an electrode and a standard electrolyte solution.

2. The microchip as set forth in claim 1, further comprising an impedance analyzer to which the electrodes housed in the first and the second reservoirs are electrically connected, the impedance analyzer detecting an impedance change resulted from interference of the movement of the anions that are contained in the first standard electrolyte solution and move across the micro-channel from the first polyelectrolytic salt bridge to the second polyelectrolytic salt bridge, by the sample moving along the micro-channel.

3. The microchip as set forth in claim 2, wherein peak amplitude of the impedance change is dependent upon the size of the sample.

4. The microchip as set forth in claim 1, wherein the microchip comprises two electrode systems separated by a fixed length in the micro-channel to detect velocity of the moving sample, based on the fixed length and a time elapsed at which the impedance change has occurred at each of the two electrode systems.

5. The microchip as set forth in claim 1, wherein the electrode is a metal/metal salt electrode.

6. The microchip as set forth in claim 1, wherein the polyelectrolytic salt bridge is formed by photo-polymerization.

7. The microchip as set forth in claim 1, wherein the polyelectrolytic salt bridge is formed from poly-diallyldimethyl ammonium chloride.

8. The microchip as set forth in claim 1, comprising:
a) an inlet through which a sample solution containing two or more cells is introduced;
b) a micro-channel along which the sample solution moves;
c) two or more outlets which discharge the sample solution passed through the micro-channel;
d) a first electrode system comprising
i) a first and a second polyelectrolytic salt bridges, each of which is oppositely connected to the micro-channel and
ii) a first and a second reservoirs, each of which is connected to the first and the second polyelectrolytic salt bridges and houses an electrode and a standard electrolyte solution;
e) a second electrode system comprising
i) a third and a fourth polyelectrolytic salt bridges, each of which is oppositely connected to the micro-channel and
ii) a third and a fourth reservoirs, each of which is connected to the third and the fourth polyelectrolytic salt bridges and houses an electrode and a standard electrolyte solution; and
f) a means for converting a moving direction of the sample solution.

9. The microchip as set forth in claim 8, wherein the microchip comprises two outlets in which a first outlet is located on an extended line from the microchannel to collect cells of no concern and a second outlet is located at out of the extended line to collect a cell of concern by working of the means for converting a movement direction of the sample solution.

10. The microchip as set forth in claim 1, wherein the microchip is used for cell counting, cell velocimetry or cell sorting.
